(19) United States
(12) Patent Application Publication
(10) Pub. No.: US 2006/0211020 A1
(43) Pub. Date: Sep. 21, 2006

(54) Methods for the diagnosis, prognosis and treatment of metabolic syndrome

(75) Inventors: Lindsay A. Farrer, Ashland, MA (US); Diego F. Wyszynski, Sharon, MA (US)

Correspondence Address: Ronald I. Eisenstein, Esq.;NIXON PEABODY LLP, 100 Summer Street, Boston, MA, 02110, US

(73) Assignee: The Trustees of Boston University, Boston, MA (US)

(21) Appl. No.: 11/359,346

(22) Filed: Feb. 22, 2006

Related U.S. Application Data
(63) Continuation of application No. PCT/US04/27758, filed on Aug. 26, 2004.
(60) Provisional application No. 60/498,082, filed on Aug. 26, 2003.

(51) Int. Cl. C12Q 1/68; A61K 39/395; A61K 38/16; A61K 38/17; A61K 48/00

(52) U.S. Cl. 435/6; 424/146.1; 514/12; 514/44

(57) ABSTRACT

The present invention provides methods for detecting susceptibility to metabolic syndrome. In particular, the presence of differences in at least one of the following genes; microsomal triglyceride transfer protein (MTP), fatty acid binding protein 2 (FABP2), annexin A5 (ANXA5), pyruvate dehydrogenase (lipoamide) alpha 2 (PDHA2), CDP-diacylglycerol synthase (phosphatidate cytidylyltransferase) 1 (CDS 1), and glycerol kinase 2 (GK2) serves as a prognostic and diagnostic indicator of metabolic syndrome. Furthermore, metabolic syndrome can be treated by regulating the levels of MTP, FABP2, ANXA5, PDHA2, CDS1, and GK2.

### CROSS-REFERENCE TO RELATED APPLICATION

This application claims the benefit under 35 U.S.C. 119(e) of U.S. Provisional Patent Application Ser. No. 60/498,082, filed Aug. 26, 2003.

### FIELD OF THE INVENTION

The present invention relates to diagnostic and prognostic tests for the detection of certain genes predisposing individuals to metabolic syndrome. In addition, it relates to a treatment method for metabolic syndrome.

### BACKGROUND

Obesity associated with hypertension, glucose intolerance, atherosclerosis, and dyslipidemia is also known as metabolic syndrome or syndrome X. Due to complexity of the phenotype, high prevalence of individual phenotypic components in the population and a number of environmental factors affecting the phenotype, identifying the genetic predisposition factors of metabolic syndrome has been complicated.

Genome wide scans have identified at least two loci that are associated with the metabolic syndrome: a quantitative trait locus on 3q27 and 17p12. The pedigree-based analysis indicated that a locus on 3q27 is associated with weight, waist circumference, leptin levels, insulin levels, insulin/glucose ratio and hip circumference. The locus on 17p12 was strongly linked to plasma leptin levels. Both these chromosomal regions contain a number of genes that can be considered as candidate genes for the metabolic syndrome. A glucocorticoid receptor gene located on 5q31-q33 has been suggested to be associated with metabolic syndrome or some phenotypic components thereof (U.S. Pat. Nos. 6,156,510). However, polymorphisms in this loci do not seem to be associated with all the phenotypic components of the metabolic syndrome. Therefore, it is likely that more susceptibility loci exist.

Early determination of susceptibility is extremely important because of the environmental effects on metabolic syndrome. By knowing the possibility of such susceptibility, an individual can change diet, life style, etc. to reduce the risk of onset and/or severity of metabolic syndrome. Additionally, knowledge of such risk of susceptibility permits earlier medical intervention.

To enhance the accurate genetic diagnosis to allow early identification of individuals at risk or already suffering from the metabolic syndrome, it would be advantageous to identify further genetic factors predisposing one to this complicated disorder.

Identification of the genetic factors predisposing to the metabolic syndrome would also allow development of more targeted therapeutic interventions. At present, the only available treatments for these disorders are agents that are not targeted to an individual's actual defect but rather to the various symptoms. Examples include ACE inhibitors and diuretics for hypertension, insulin supplementation for glucose intolerance, cholesterol reduction strategies for dyslipidemia, anti-coagulants, and β-blockers for cardiovascular disorders, and a number of weight reduction drugs.

Thus, it is important to locate additional and more accurate markers that are predictive and diagnostic of metabolic syndrome. In addition, these markers may provide for a more targetd therapeutic approach to treatment.

### SUMMARY OF THE INVENTION

We have now discovered a new method for detection of susceptibility to metabolic syndrome. This method involves analyzing certain genes that map to human chromosome 4q between the markers D4S2361 and D4S2394 for a polymorphism or other nucleic acid change linked to metabolic syndrome. Preferably, the polymorphism or other change is in at least one of microsomal triglyceride transfer protein (MTP) gene, the fatty acid binding protein 2 (FABP2) gene, the annexin A5 (ANXA5) gene, the pyruvate dehydrogenase (lipoamide) alpha 2 (PDHA2) gene, the CDP-diacylglycerol synthase (phosphatidate cytidylyltransferase) 1 (CDS1) gene, or the glycerol kinase 2 (GK2) gene.

In one embodiment of the present invention, the polymorphism is a short tandem repeat (STR). Alternatively, the polymorphism is a single nucleotide polymorphism (SNP). The change can also be a modification to the nucleic acid that effects expression, such as, for example, methylation. For one embodiment, the promoter is methylated.

In a preferred embodiment, a method for the diagnosis and prognosis of metabolic syndrome is disclosed. The method involves obtaining a sample from an individual, such as DNA or RNA, and determining the presence or absence of a nucleic acid difference (i.e. a polymorphism) in at least one, preferably all of the microsomal triglyceride transfer protein (MTP) gene, the fatty acid binding protein 2 (FABP2) gene, the annexin A5 (ANXA5) gene, the pyruvate dehydrogenase (lipoamide) alpha 2 (PDHA2) gene, the CDP-diacylglycerol synthase (phosphatidate cytidylyltransferase) 1 (CDS1) gene, or the glycerol kinase 2 (GK2) genes on chromosome 4. The presence of such a difference, as compared to a wild type sample (i.e. individuals unaffected by metabolic syndrome), is linked to metabolic syndrome and is correlated with an the prognosis and/or diagnosis of metabolic syndrome.

In another embodiment one uses a test where one looks at the expression product of at least one, preferably all of the microsomal triglyceride transfer protein (MTP) gene, the fatty acid binding protein 2 (FABP2) gene, the annexin A5 (ANXA5) gene, the pyruvate dehydrogenase (lipoamide) alpha 2 (PDHA2) gene, the CDP-diacylglycerol synthase (phosphatidate cytidylyltransferase) 1 (CDS1) genes (proteins) to determine differences between the tested individuals and wild type controls.

The DNA or RNA sample of the individual may be obtained from a white blood cell. Alternatively, the DNA or RNA sample is obtained from surgically-removed tissue. Most preferably, the tissue is adipose tissue.

In a further embodiment, methods for the treatment of metabolic syndrome are disclosed. In these methods, a patient in need of treatment is administered an agent or compound that regulates the activity of MTP, FABP2, ANXA5, PDHA2, CDS1 or GK2. The agent or compound that regulates the activity of MTP, FABP2, ANXA5, PDHA2, CDS1, or GK2 may be an inhibitor. Alternatively, the agent or compound that regulates the activity of MTP, FABP2, ANXA5, PDHA2, CDS1, or GK2 may be an activator.

The inhibitor may be an antibody, small molecule, antisense nucleic acid, RNAi, siRNA, PNA, or aptamer. The activator may be a small molecule, partial agonists, inverse agonist, activator, or co-activator.

In one embodiment, one would look for such differences in an individual related to an individual diagnosed as having metabolic syndrome.

In another embodiment the test can be used for diagnosis, prognosis, treatment and/or classification of the metabolic syndrome.

In yet another embodiment, we disclose a kit for the diagnosis and/or prognosis of metabolic syndrome.

### BRIEF DESCRIPTION OF THE FIGURES

FIGS. 1A-1D show four sub-families of a large, 170 member Turkish family with more than one metabolic syndrome patient. Circles are females and squares are males; filled areas mark affected individuals. The question mark indicates individuals with unknown affection status. Affection is defined as triglyceride (TG) levels equal or greater than the 90^{th} percentile for age and sex, HDL equal or lower than 30 mg/dL for males and 34 mg/dL for females, and BMI≦30 kg/m². Unaffected individuals are those with TG levels equal or lower the 50^{th} percentile for their age and sex and HDL≧37 mg/dL for men and ≧42 mg/dL for women and BMI≦30 kg/m² and normoglycemic. Of unknown affection status are those individuals without laboratory values, those with TGs between the 50^{th} and the 90^{th} percentile, and those with HDL levels between the affected and unaffected status.

### DETAILED DESCRIPTION OF THE INVENTION

We have found that differences in genes within chromosome 4, flanked by the genetic markers D4S2391 and D4S2394, such as polymorphisms, predict susceptibility to metabolic syndrome. This 35 cM interval includes about 83 genes (see Table 1), all of which may be used to analyze nucleic acid differences (i.e. polymorphisms) associated with metabolic syndrome. We have now discovered that differences, including nucleic acid changes or modifications to the nucleic acid such as methylation, in at least one of the following 6 genes: microsomal triglyceride transfer protein (MTP), fatty acid binding protein 2 (FABP2), annexin A5 (ANXA5), pyruvate dehydrogenase (lipoamide) alpha 2 (PDHA2), CDP-diacylglycerol synthase (phosphatidate cytidylyltransferase) 1 (CDS1), and glycerol kinase 2 (GK2) are prognostic and diagnostic of metabolic syndrome.

Thus, the present invention discloses methods for predicting susceptibility to and diagnosing individuals with metabolic syndrome by analyzing at least one, preferably all of, MTP, FABP2, ANXA5, PDHA2, CDS1, and GK2 genes and/or their gene product (protein). In addition, monitoring the levels and/or activity of at least one, preferably all of, MTP, FABP2, ANXA5, PDHA2, CDS1, and GK2 proteins serves as a diagnostic and/or prognostic indicator of metabolic syndrome. Furthermore, metabolic syndrome can be treated by regulating the levels of MTP, FABP2, ANXA5, PDHA2, CDS1, and GK2.

Microsomal Triglyceride Transfer Protein (MTP)

MTP encodes the large subunit of the heterodimeric microsomal triglyceride transfer protein, and maps to chromosome 4q22-q24. The coding sequence (NCBI Locus: NM_{—}000253) is:

(SEQ ID. NO. 5)
atgattcttc ttgctgtgct ttttctctgc ttcatttcct catattcagc ttctgttaaa
ggtcacacaa ctggtctctc attaaataat gaccggctgt acaagctcac gtactccact
gaagttcttc ttgatcgggg caaaggaaaa ctgcaagaca gcgtgggcta ccgcatttcc
tccaacgtgg atgtggcctt actatggagg aatcctgatg gtgatgatga ccagttgatc
caaataacga tgaaggatgt aaatgttgaa aatgtgaatc agcagagagg agagaagagc
atcttcaaag gaaaaagccc atctaaaata atgggaaagg aaaacttgga agctctgcaa
agacctacgc tccttcatct aatccatgga aaggtcaaag agttctactc atatcaaaat
gaggcagtgg ccatagaaaa tatcaagaga ggtctggcta gcctatttca gacacagtta
agctctggaa ccaccaatga ggtagatatc tctggaaatt gtaaagtgac ctaccaggct
catcaagaca aagtgatcaa aattaaggcc ttggattcat gcaaaatagc gaggtctgga
tttacgaccc caaatcaggt cttgggtgtc agttcaaaag ctacatctgt caccacctat
aagatagaag acagctttgt tatagctgtg cttgctgaag aaacacacaa ttttggactg
aatttcctac aaaccattaa ggggaaaata gtatcgaagc agaaattaga gctgaagaca
accgaagcag gcccaagatt gatgtctgga aagcaggctg cagccataat caaagcagtt
gattcaaagt acacggccat tcccattgtg gggcaggtct tccagagcca ctgtaaagga
tgtccttctc tctcggagct ctggcggtcc accaggaaat acctgcagcc tgacaacctt
tccaaggctg aggctgtcag aaacttcctg gccttcattc agcacctcag gactgcgaag
aaagaagaga tccttcaaat actaaagatg gaaaataagg aagtattacc tcagctggtg
gatgctgtca cctctgctca gacctcagac tcattagaag ccattttgga ctttttggat
ttcaaaagtg acagcagcat tatcctccag gagaggtttc tctatgcctg tggatttgct
tctcatccca atgaagaact cctgagagcc ctcattagta agttcaaagg ttctattggt
agcagtgaca tcagagaaac tgttatgatc atcactggga cacttgtcag aaagttgtgt
cagaatgaag gctgcaaact caaagcagta gtggaagcta agaagttaat cctgggagga
cttgaaaaag cagagaaaaa agaggacacc aggatgtatc tgctggcttt gaagaatgcc
ctgcttccag aaggcatccc aagtcttctg aagtatgcag aagcaggaga agggcccatc
agccacctgg ctaccactgc tctccagaga tatgatctcc ctttcataac tgatgaggtg
aagaagacct taaacagaat ataccaccaa aaccgtaaag ttcatgaaaa gactgtgcgc
actgctgcag ctgctatcat tttaaataac aatccatcct acatggacgt caagaacatc
ctgctgtcta ttggggagct tccccaagaa atgaataaat acatgctcgc cattgttcaa
gacatcctac gtttggaaat gcctgcaagc aaaattgtcc gtcgagttct gaaggaaatg
gtcgctcaca attatgaccg tttctccagg agtggatctt cttctgccta cactggctac
atagaacgta gtccccgttc ggcatctact tacagcctag acattctcta ctcgggttct
ggcattctaa ggagaagtaa cctgaacatc tttcagtaca ttgggaaggc tggtcttcac
ggtagccagg tggttattga agcccaagga ctggaagcct taatcgcagc cacccctgac
gagggggagg agaaccttga ctcctatgct ggtatgtcag ccatcctctt tgatgttcag
ctcagacctg tcaccttttt caacggatac agtgatttga tgtccaaaat gctgtcagca
tctggcgacc ctatcagtgt ggtgaaagga cttattctgc taatagatca ttctcaggaa
cttcagttac aatctggact aaaagccaat atagaggtcc agggtggtct agctattgat
atttcaggtg caatggagtt tagcttgtgg tatcgtgagt ctaaaacccg agtgaaaaat
agggtgactg tggtaataac cactgacatc acagtggact cctcttttgt gaaagctggc
ctggaaacca gtacagaaac agaagcaggc ttggagttta tctccacagt gcagttttct
cagtacccat tcttagtttg catgcagatg gacaaggatg aagctccatt caggcaattt
gagaaaaagt acgaaaggct gtccacaggc agaggttatg tctctcagaa aagaaaagaa
agcgtattag caggatgtga attcccgctc catcaagaga actcagagat gtgcaaagtg
gtgtttgccc ctcagccgga tagtacttcc agcggatggt tttga

Microsomal triglyceride transfer protein catalyzes the transport of triglyceride, cholesteryl ester, and phospholipid between phospholipid surfaces. It is a heterodimer composed of a 55-kD multifunctional protein, protein disulfide isomerase (PDI), and a unique large subunit with an apparent molecular weight of 88 kD (Wetterau et al., 1990). MTP was isolated as a soluble protein from the lumen of a microsomal fraction of liver and intestine.

Ohashi et al. (2000) stated that 14 separate mutations in the MTP gene and/or cDNA from patients with abetalipoproteinemia (ABL) had been described. They identified MTP mutations in all 8 alleles of 2 Japanese and 2 American patients with ABL.

According to the methods of the present invention, differences present in the MTP gene or gene product are diagnostic and/or prognostic of metabolic syndrome. MTP may be analyzed alone or in combination with any one of FABP2, ANXA5, PDHA2, CDS1, and GK2.

Fatty Acid Binding Protein 2 (FABP2)

FABP2, also known as FABPI, is located on chromosome 4 and maps to 4q28-q31. The coding sequence (NCBI Locus: NM_{—}000134) is:

(SEQ ID. NO. 6)
atggcgtttg acagcacttg gaaggtagac cggagtgaaa actatgacaa gttcatggaa
aaaatgggtg ttaatatagt gaaaaggaag cttgcagctc atgacaattt gaagctgaca
attacacaag aaggaaataa attcacagtc aaagaatcaa gcgcttttcg aaacattgaa
gttgtttttg aacttggtgt cacctttaat tacaacctag cagacggaac tgaactcagg
gggacctgga gccttgaggg aaataaactt attggaaaat tcaaacggac agacaatgga
aacgaactga atactgtccg agaaattata ggtgatgaac tagtccagac ttatgtgtat
gaaggagtag aagccaaaag gatctttaaa aaggattga

The intracellular fatty acid-binding proteins (FABPs) belong to a multigene family with nearly twenty identified members. FABPs are divided into at least three distinct types, namely the hepatic-, intestinal- and cardiac-type. They form 14-15 kDa proteins and are thought to participate in the uptake, intracellular metabolism and/or transport of long-chain fatty acids. They may also be responsible in the modulation of cell growth and proliferation. Intestinal fatty acid-binding protein 2 gene contains four exons and is an abundant cytosolic protein in small intestine epithelial cells. This gene has a polymorphism at codon 54 that identified an alanine-encoding allele and a threonine-encoding allele. Thr-54 protein is associated with increased fat oxidation and insulin resistance.

According to the methods of the present invention, differences present in the FABP2 gene or gene product are diagnostic and/or prognostic of metabolic syndrome. FABP2 may be analyzed alone or in combination with any one of MTP, ANXA5, PDHA2, CDS1, and GK2.

Annexin A5 (ANXA5)

Annexin A5 (ANXA5), also known as PP4, ANX5, ENX2, ANNEXIN V, ENDONEXIN II, PLACENTAL ANTICOAGULANT PROTEIN I, VASCULAR ANTICOAGULANT-ALPHA, LIPOCORTIN V, PLACENTAL PROTEIN 4, and ANCHORIN CII, is located on chromosome 4 and maps to 4q26-q28; 4q28-q32. The coding sequence (NCBI Locus: NM_{—}001154) is:

(SEQ ID. NO. 7)
atggcacagg ttctcagagg cactgtgact gacttccctg gatttgatga gcgggctgat
gcagaaactc ttcggaaggc tatgaaaggc ttgggcacag atgaggagag catcctgact
ctgttgacat cccgaagtaa tgctcagcgc caggaaatct ctgcagcttt taagactctg
tttggcaggg atcttctgga tgacctgaaa tcagaactaa ctggaaaatt tgaaaaatta
attgtggctc tgatgaaacc ctctcggctt tatgatgctt atgaactgaa acatgccttg
aagggagctg gaacaaatga aaaagtactg acagaaatta ttgcttcaag gacacctgaa
gaactgagag ccatcaaaca agtttatgaa gaagaatatg gctcaagcct ggaagatgac
gtggtggggg acacttcagg gtactaccag cggatgttgg tggttctcct tcaggctaac
agagaccctg atgctggaat tgatgaagct caagttgaac aagatgctca ggctttattt
caggctggag aacttaaatg ggggacagat gaagaaaagt ttatcaccat ctttggaaca
cgaagtgtgt ctcatttgag aaaggtgttt gacaagtaca tgactatatc aggatttcaa
attgaggaaa ccattgaccg cgagacttct ggcaatttag agcaactact ccttgctgtt
gtgaaatcta ttcgaagtat acctgcctac cttgcagaga ccctctatta tgctatgaag
ggagctggga cagatgatca taccctcatc agagtcatgg tttccaggag tgagattgat
ctgtttaaca tcaggaagga gtttaggaag aattttgcca cctctcttta ttccatgatt
aagggagata catctgggga ctataagaaa gctcttctgc tgctctgtgg agaagatgac
taa

The protein encoded by this gene belongs to the annexin family of calcium-dependent phospholipid binding proteins some of which have been implicated in membrane-related events along exocytotic and endocytotic pathways. Annexin 5 is a phospholipase A2 and protein kinase C inhibitory protein with calcium channel activity and plays a potential role in cellular signal transduction, inflammation, and growth and differentiation. The gene spans 29 kb containing 13 exons, and encodes a single transcript of approximately 1.6 kb and a protein product with a molecular weight of about 35 kDa.

According to the methods of the present invention, differences present in the ANXA5 gene or gene product are diagnostic and/or prognostic of metabolic syndrome. ANXA5 may be analyzed alone or in combination with any one of MTP, FABP2, PDHA2, CDS1, and GK2.

Pyruvate dehydrogenase (lipoamide) alpha 2 (PDHA2)

PDHA2 is located on chromosome 4 and maps to 4q22-q23. The coding sequence (NCBI Locus: NM_{—}005390) is: (SEQ. ID. NO. 4)

(SEQ ID. NO. 8)
atgctggccg ccttcatctc ccgcgtgttg aggcgagttg cccagaaatc agctcgcaga
gtgctggtgg catcccgtaa ctcctcaaat gacgctacat ttgaaattaa gaaatgtgat
ctttatctgt tggaagaggg tccccctgtc actacagtgc tcactagggc ggaggggctt
aaatactaca ggatgatgct gactgttcgc cgcatggaat tgaaggcaga tcagctgtac
aaacagaaat tcattcgcgg tttctgtcac ctgtgcgatg gtcaggaagc ttgttgcgtg
ggccttgagg ccggcataaa cccctcggat cacgtcatta catcctatag ggctcatggt
gtgtgctata ctcggggact ttctgtccga tccattctcg cagagctgac gggaagaaga
ggaggttgtg ctaaaggaaa aggaggatcg atgcatatgt ataccaagaa cttctatggg
ggcaatggca tcgtcggtgc acagggcccc ctgggcgctg gcattgctct ggcctgtaaa
tataaaggaa acgatgagat ctgtttgact ttatatgggg atggcgctgc gaatcagggg
cagatagccg aagctttcaa tatggcagct ttatggaaat taccttgtgt tttcatctgt
gagaataacc tatatggaat gggaacatct actgagagag cagcagccag ccctgattac
tacaagaggg gcaattttat ccctgggcta aaggtcgatg gaatggatgt tctgtgtgtt
cgtgaggcaa caaaatttgc agctaactac tgtagatctg gaaaggggcc catactgatg
gagctgcaaa cctaccgtta tcatggacac agtatgagtg atcctggagt cagttatcgt
acacgagaag aaattcagga agtaagaagt aagagggatc ctataataat tctccaagat
agaatggtaa acagcaagct cgccactgtg gaagaattaa aggaaattgg ggctgaggtg
aggaaagaaa ttgatgatgc tgcccagttt gctaccactg atcctgagcc acatttggaa
gaattaggcc atcacatcta cagcagtgat tcatcttttg aagttcgtgg tgcaaatcca
tggatcaagt ttaagtccgt cagttaa

The pyruvate dehydrogenase (PDH) complex converts pyruvate to acetyl CoA, an essential step in aerobic glucose metabolism. Dahl et al. (1990) extended their previous work on the gene for the E1-alpha subunit of this complex, expressed in somatic tissues and located on band Xp22.1. Using the probe for the X-linked gene, they found significant in situ hybridization with an autosomal locus, PDHA2, located on 4q22-q23. DNA sequencing of the gene showed that the transcribed region spans only approximately 1.4 kb.

According to the methods of the present invention, differences present in the PDHA2 gene or gene product are diagnostic and/or prognostic of metabolic syndrome. PDHA2 may be analyzed alone or in combination with any one of MTP, FABP2, ANXA5, CDS1, and GK2.

CDP-diacylglycerol Synthase (Phosphatidate Cytidylyltransferase) 1 (CDS1)

CDP-diacylglycerol synthase (phosphatidate cytidylyltransferase) 1 (CDS1), also known as CDS, is located on chromosome 4 and maps to 4q21.23. The coding sequence (NCBI Locus: NM_{—}001263) is:

(SEQ ID. NO. 9)
atgttggagc tgaggcaccg gggaagctgc cccggcccca gggaagcggt gtcgccgcca
caccgcgagg gagaggcggc cggcggcgac cacgaaaccg agagcaccag cgacaaagaa
acagatattg atgacagata tggagatttg gattccagaa cagattctga tattccggaa
attccaccat cctcagatag aacccctgag attctcaaaa aagctctatc tggtttatct
tcaaggtgga aaaactggtg gatacgtgga attctcactc taactatgat ctcgttgttt
ttcctgatca tctatatggg atccttcatg ctgatgcttc ttgttctggg catccaagtg
aaatgcttcc atgaaattat cactataggt tatagagtct atcattctta tgatctacca
tggtttagaa cactaagttg gtactttcta ttgtgtgtaa actacttttt ctatggagag
actgtagctg attattttgc tacatttgtt caaagagaag aacaacttca gttcctcatt
cgctaccata gatttatatc atttgccctc tatctggcag gtttctgcat gtttgtactg
agtttggtga agaaacatta tcgtctgcag ttttatatgt tcgcatggac tcatgtcact
ttactgataa ctgtcactca gtcacacctt gtcatccaaa atctgtttga aggcatgata
tggttccttg ttccaatatc aagtgttatc tgcaatgaca taactgctta cctttttgga
tttttttttg ggagaactcc attaattaag ttgtctccta aaaagacttg ggaaggattc
attggtggtt tcttttccac agttgtgttt ggattcattg ctgcctatgt gttatccaaa
taccagtact ttgtctgccc agtggaatac cgaagtgatg taaactcctt cgtgacagaa
tgtgagccct cagaactttt ccagcttcag acttactcac ttccaccctt tctaaaggca
gtcttgagac aggaaagagt gagcttgtac cctttccaga tccacagcat tgcactgtca
acctttgcat ctttaattgg cccatttgga ggcttctttg ctagtggatt caaaagagcc
ttcaaaatca aggattttgc aaataccatt cctggacatg gtgggataat ggacagattt
gattgtcagt atttgatggc aacttttgta catgtgtaca tcacaagttt tatccggggc
ccaaatccca gcaaagtgct acagcagttg ttggtgcttc aacctgaaca gcagttaaat
atatataaaa ccctgaagac tcatctcatt gagaaaggaa tcctacaacc caccttgaag
gtataa

By searching EST databases for sequences related to Drosophila CDS, Heacock et al. (1996) and Weeks et al. (1997) identified cDNAs encoding a human CDS, CDS1. Heacock et al. (1996) reported that the deduced 444-amino acid human CDS1 protein shares 50%, 37%, and 31% identity with Drosophila, S. cerevisiae, and E. coli CDS proteins, respectively. Sequence analysis indicated that the human enzyme contains 3 putative membrane-spanning domains. Using Northern blot analysis, Heacock et al. (1996) determined that CDS1 was expressed as an approximately 5-kb mRNA in all human tissues examined, with the most prominent expression in heart and liver. An additional 3-kb transcript was present in heart and pancreas. Weeks et al. (1997) reported a CDS1 cDNA sequence that differed from that determined by Heacock et al. (1996) primarily in the 3-prime coding sequence and the 3-prime untranslated region. Weeks et al. (1997) demonstrated that human CDS1 can complement a yeast cds1 null mutant strain.

According to the methods of the present invention, differences present in the CDS1 gene or gene product are diagnostic and/or prognostic of metabolic syndrome. CDS1 may be analyzed alone or in combination with any one of MTP, FABP2, ANXA5, PDHA2, and GK2.

Glycerol Kinase 2 (GK2)

Glycerol Kinase 2 (GK2), also known as GKTA, GLYCEROL KINASE PSEUDOGENE 2, and GKP2 is located on chromosome 4 and maps to 4q13. The coding sequence (NCBI Locus: NM_{—}033214) is:

(SEQ ID. NO. 10)
atggcagccc caaagacagc agctgtgggg ccgttggtgg gagcggtggt ccagggcacc
aactccactc gctttctggt tttcaattca aaaacagcgg aactacttag tcatcacaaa
gtggaattaa cacaagagtt cccaaaagaa ggatgggtgg aacaagaccc taaagaaatt
cttcagtctg tctacgagtg tatagcgaga acgtgtgaga aacttgacga actgaatatt
gatatatcca acataaaagc tgttggtgtc agcaatcaga gggaaaccac tgtaatctgg
gacaagttaa caggagagcc tctctacaat gctgtggtgt ggcttgatct aagaacccag
actactgttg aggatcttag taaaaaaatt ccaggaaata gtaacttcgt caagtctaag
acaggccttc cactcagcac ttacttcagt gcagtaaaac ttcgttggat gcttgacaat
gtgagaaacg tccaaaaggc tgttgaagaa ggtagagctc tttttggtac cattgattca
tggcttatct ggagtttgac aggaggagtt aatggaggcg tgcattgtac agatgtaaca
aatgcaagta ggacaatgct ttttaatatc cattctttgg aatgggataa agagctctgt
gacttttttg aaattccaat ggaccttctt ccaaatgtct tcagttcttc tgagatctat
ggcctaatta aaactggagc cctggaaggt gtgccaatat ctgggtgttt gggggaccaa
tgtgctgcat tagtaggaca aatgtgcttc caggagggac aagccaaaaa cacctatgga
acaggttgct tcttactgtg taatacgggt cgtaaatgtg tgttttctga acatggcctt
ttgaccacag tagcttacaa actaggcaga gagaagccag catattatgc actggaaggt
tctgttgcta tagcaggtgc tgttattcgt tggctaagag acaatcttgg aattatagag
acctcaggag acattgaaag acttgctaaa gaagtaggaa cttcttatgg ctgttacttt
gtcccagcct tttcagggtt atatgcacct tattgggagc ccagtgcaag agggatactc
tgtggcctca ctcagtttac caataaatgt catattgctt ttgctgcatt agaagctgtt
tgtttccaaa cccgagagat tttggaagcc atgaaccgtg actgtggaat tccacttcgt
catttgcagg tagatggagg aatgaccaac aacaaagttc ttatgcagct acaagcagat
attcttcata ttccagtaat aaaacccttt atgcctgaaa caactgcact aggagctgcc
atggcagcag gggctgcaga gggagtaagc gtttggagcc ttgaacccca ggctttgtca
gttctcagga tggaacgatt tgaaccacag atccaggcca cagaaagtga aattcgttat
gccacatgga agaaagccgt aatgaagtca atgggttggg ttaccagtca gtctcctgaa
ggtggtgatc cttctatctt ctctagtctg cctttgggat tttttatagt gagtagcatg
gtaatgctaa ttggagcaag atatatctcg ggtgtgccat aa

Sargent et al. (1994) suggested that the human glycerol kinase gene family consists of at least 3 expressed loci. The GK1 locus on Xp21.3 is the site of mutations (deletions) causing glycerol kinase deficiency. It comprises 19 exons and is probably ancestral to several other genes which, because they are intronless, are suspected of having arisen by reverse transcriptase mediated events. These include 2 genes on chromosome 4. They are expressed as a single mRNA species in testis where expression is at a high level. By fluorescence in situ hybridization, Sargent et al. (1994) demonstrated that one of the testicular forms of GK is encoded by a gene at 4q13 and the other by a gene at 4q32.

According to the methods of the present invention, differences present in the GK2 gene or gene product are diagnostic and/or prognostic of metabolic syndrome. GK2 may be analyzed alone or in combination with any one of MTP, FABP2, ANXA5, PDHA2, and CDS1.

Detection of Changes

Differences in the MTP, FABP2, ANXA5, PDHA2, CDS1, and GK2 genes between individuals susceptible to metabolic syndrome and individuals not affected (referred to as wild type) can be detected by any method known to those of skill in the art. For example, there are several methods that can be used to detect DNA or RNA sequence variation, all of which are encompassed by the methods of the present invention. Such tests are commonly performed using DNA or RNA collected from biological samples, e.g., tissue biopsies, urine, stool, sputum, blood, cells, tissue scrapings, breast aspirates or other cellular materials, and can be performed by a variety of methods including, but not limited to, PCR, hybridization with allele-specific probes, enzymatic mutation detection, chemical cleavage of mismatches, mass spectrometry or DNA sequencing, including minisequencing. These differences are then compared with a collection of sequences from wild type individuals. One can also look at modifications to the nucleic acid that effect expression such as methylation.

One can also look for changes by using a probe for gene production. Changes in the nucleic acid that will have an effect on gene expression are those that truncate the gene product or prevent its normal expression. Thus, using an antibody as a probe can result in a quick test to determine if an individual is at risk for, or currently has, metabolic syndrome. Preferably, the antibody is to the C-terminal end of the protein (gene product).

The test can be carried out prenatally (on amnio-cytes, fetal cells in maternal blood or chorionic villi), or presymptomatically (from bucchal sample or white blood cells) in young or adult individuals. It can also be performed on archival tissues, or on tissues removed for biopsy. In a preferred embodiment, the DNA or RNA is collected from blood cells. Alternatively, the DNA or RNA is collected from tissue such as adipose tissue.

Since there is a genetic link to metabolic syndrome, one preferred population to test for metabolic syndrome susceptibility are family members of an individual diagnosed with metabolic syndrome. Preferably one would look at family members up to the seventh degree distant from the metabolic syndrome individual. Another preferred grouping would be family members up to the sixth degree distant from the metabolic syndrome individual. More preferably, one would look at individuals up to the fifth degree distant from the metabolic syndrome individual. Still more preferably, one would look at individuals up to the fourth degree distant from the metabolic syndrome individual. Even more preferably one would look at individuals up to the third degree distant from the metabolic syndrome individual. Yet more preferably one would look at individuals up to the second degree distant from the metabolic syndrome individual.

In particular embodiments, changes are detected on a solid phase support. Hybridization with allele specific probes can be conducted in two formats: (1) allele specific oligonucleotides bound to a solid phase (glass, silicon, nylon membranes) and the labeled sample in solution, as in many DNA chip applications, or (2) bound sample (often cloned DNA or PCR amplified DNA) and labeled oligonucleotides in solution (either allele specific or short so as to allow sequencing by hybridization). Diagnostic and prognostic tests may involve a panel of variances, often on a solid support, which enables the simultaneous determination of more than one variance. Thus, in one embodiment each of the MTP, FABP2, ANXA5, PDHA2, CDS1, and GK2 genes are analyzed on one or by one solid support. Alternatively, one or any number of the 6 genes (MTP, FABP2, ANXA5, PDHA2, CDS1, and GK2) may be analyzed by the method of the present invention. Methods for such diagnostic tests are well known in the art and disclosed in patent application WO 00/04194, incorporated herein by reference.

Types of probe useful in the present invention include cDNA, riboprobes, synthetic oligonucleotides, genomic probes, or antibodies (for gene product detection). The type of probe used will generally be dictated by the particular situation, such as riboprobes for in situ hybridization, and cDNA for Northern blotting, for example. Most preferably, the probe is directed to nucleotide regions unique to the protein. Detection of the MTP, FABP2, ANXA5, PDHA2, CDS1, and GK2 encoding genes, per se, will be useful in screening for nucleic acid changes. Other forms of assays to detect targets more readily associated with levels of expression, transcripts and other expression products, will generally be useful as well. The probes may be as short as is required to differentially recognize MTP, FABP2, ANXA5, PDHA2, CDS1, and GK2 mRNA transcripts (as compared to wild type controls), and may be as short as, for example, 15 bases; however, probes of at least 17 bases, more preferably 18 bases and still more preferably 20 bases are preferred.

A probe may also be reverse-engineered by one skilled in the art from the amino acid sequence of the MTP, FABP2, ANXA5, PDHA2, CDS1, and GK2. However, use of such probes may be more limited than the native DNA sequence, as it will be appreciated that any one given reverse-engineered sequence will not necessarily hybridize well, or at all, with any given complementary sequence reverse-engineered from the same peptide, owing to the degeneracy of the genetic code. This is a factor common in the calculations of those skilled in the art, and the degeneracy of any given sequence is frequently so broad as to yield a large number of probes for any one sequence.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

Direct DNA sequencing, either manual sequencing or automated fluorescent sequencing can detect sequence variation. For large genes, manual sequencing is very labor-intensive, but under optimal conditions, mutations in the coding sequence of a gene are rarely missed. Another approach is the single-stranded conformation polymorphism assay (SSCA) (Orita et al., 1989). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection. The fragments which have shifted mobility on SSCA gels may then be sequenced to determine the exact nature of the DNA sequence variation. Other approaches based on the detection of mismatches between the two complementary DNA strands include clamped denaturing gel electrophoresis (CDGE) (Sheffield et al., 1991), heteroduplex analysis (HA) (White et al., 1992) and chemical mismatch cleavage (CMC) (Grompe et al., 1989). None of the methods described above will detect large deletions, duplications or insertions, nor will they detect a regulatory mutation which affects transcription or translation of the protein. Other methods which might detect these classes of mutations such as a protein truncation assay or the asymmetric assay, detect only specific types of mutations and would not detect missense mutations. A review of currently available methods of detecting DNA sequence variation can be found in a review by Grompe (1993). An allele specific detection approach such as allele specific oligonucleotide (ASO) hybridization can be utilized to rapidly screen large numbers of other samples for that same mutation.

A rapid preliminary analysis to detect changes in DNA sequences can be performed by looking at a series of Southern blots of DNA cut with one or more restriction enzymes, preferably with a large number of restriction enzymes. Each blot contains at least one control (i.e. DNA from a person who does not have metabolic syndrome) and at least one test sample. Southern blots displaying hybridizing fragments (differing in length from control DNA when probed with sequences near or including the MTP, FABP2, ANXA5, PDHA2, CDS1, or GK2 locus) indicate a possible mutation. If restriction enzymes which produce very large restriction fragments are used, then pulsed field gel electrophoresis (PFGE) is employed.

Detection of point mutations may be accomplished by molecular cloning of the MTP, FABP2, ANXA5, PDHA2, CDS1, or GK2 allele(s) and sequencing the allele(s) using techniques well known in the art. Alternatively, the gene sequences can be amplified directly from a genomic DNA preparation from a biological sample, using known techniques. The DNA sequence of the amplified sequences can then be determined.

In a preferred embodiment, PCR techniques are used to determine differences in the nucleotide sequence of a particular MTP, FABP2, ANXA5, PDHA2, CDS1, or GK2 allele (as compared to wild type controls). Pairs of primers are designed to be single-stranded DNA primers that can be annealed to sequences within or surrounding the MTP, FABP2, ANXA5, PDHA2, CDS1, or GK2 genes on chromosome 4. The primers aid in the amplification of DNA. The set of primers preferably allows synthesis of both intron and exon sequences. Allele-specific primers can also be used. Such primers anneal only to particular MTP, FABP2, ANXA5, PDHA2, CDS1, or GK2 mutant alleles, and thus will only amplify a product in the presence of the mutant allele as a template.

Primers useful according to the present invention are designed using amino acid sequences of the protein or nucleic acid sequences of the MTP, FABP2, ANXA5, PDHA2, CDS1, or GK2 genes. The primers are designed in the homologous regions of the gene wherein at least two regions of homology are separated by a divergent region of variable sequence, the sequence being variable either in length or nucleic acid sequence.

For example, the identical or highly, homologous, preferably at least 80%-85% more preferably at least 90-99% homologous amino acid sequence of at least about 6, preferably at least 8-10 consecutive amino acids are used to generate primers. Most preferably, the amino acid sequence is 100% identical. Forward and reverse primers are designed based upon the maintenance of codon degeneracy and the representation of the various amino acids at a given position among the known gene family members. Degree of homology as referred to herein is based upon analysis of an amino acid sequence using a standard sequence comparison software, such as protein-BLAST using the default settings (http://www.ncbi.nlm.nih.gov/BLAST/). Primers may be designed using a number of available computer programs, including, but not limited to Oligo Analyzer 3.0; Oligo Calculator; NetPrimer; Methprimer; Primer3; WebPrimer; PrimerFinder; Primer9; Oligo2002; Pride or GenomePride; Oligos; and Codehop. Detailed information about these programs can be obtained, for example, from www.molbiol.net.

Analysis of amplification products can be performed using any method capable of separating the amplification products according to their size, including automated and manual gel electrophoresis, mass spectrometry, and the like. The different alleles are identified either utilizing the difference in length or sequence of the PCR product. The length polymorphisms may be differentiated, for example using a denaturing polyacrylamide or agarose gel. The sequence polymorphisms can be differentiated using a number of methods known to one skilled in the art as described above and herein.

The methods of nucleic acid isolation, amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Moller, R. Beynon, C. Howe, Springer Verlag; 1st edition (Oct. 15, 2000), ISBN: 0387916008.

In order to facilitate subsequent cloning of amplified sequences, primers may have restriction enzyme site sequences appended to their 5′ ends. Thus, all nucleotides of the primers are derived from MTP, FABP2, ANXA5, PDHA2, CDS1, or GK2 sequences or sequences adjacent to MTP, FABP2, ANXA5, PDHA2, CDS1, or GK2, except for the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Given the sequence of the MTP, FABP2, ANXA5, PDHA2, CDS1, or GK2 open reading frames, design of particular primers is well within the skill of the art.

In a further embodiment, one could use the differential display technique to look for such nucleic acid or gene product changes.

In another embodiment, nucleic acid changes may be in genes that affect the expression of MTP, FABP2, ANXA5, PDHA2, CDS1, and GK2. In one embodiment, the change is in a promoter. Alternatively the change may be in a suppressor or activator of gene expression.

Detection of MTP, FABP2, ANXA5, PDHA2, CDS1, or GK2 Polypeptides

In one embodiment of the present application, MTP, FABP2, ANXA5, PDHA2, CDS1, and GK2 polypeptides are detected in order to predict one's susceptibility to and/or to diagnose metabolic syndrome. In the methods of the present application, the enhanced, reduced, or ablated expression of one or any number of MTP, FABP2, ANXA5, PDHA2, CDS1, and GK2 is diagnostic and/or prognostic of metabolic syndrome.

Methods for the detection of protein are well known to those skilled in the art, and include ELISA (enzyme linked immunosorbent assay), RIA (radioimmunoassay), Western blotting, and immunohistochemistry. Immunoassays such as ELISA or RIA, which can be extremely rapid, are more generally preferred. Antibody arrays or protein chips can also be employed, see for example U.S. patent application Ser. Nos: 20030013208A1; 20020155493A1, 20030017515 and U.S. Pat. Nos.: 6,329,209; 6,365,418, herein incorporated by reference in their entirety.

In one embodiment, a labeled or labelable antibody which specifically binds to MTP, FABP2, ANXA5, PDHA2, CDS1, or GK2 polypeptide is utilized. In one embodiment, the antibody is used as a probe to detect gene product. An antibody specific for the C-terminus of the gene product may detect a truncated gene product. The antibody probe may be used to detect an absence of gene product or an alteration in expression of gene product. As used herein, the phrase “labeled or labelable” refers to the attaching or including of a label (e. g., a marker or indicator) or ability to attach or include a label (e. g., a marker or indicator). Markers or indicators include, but are not limited to, for example, radioactive molecules, colorimetric molecules, and enzymatic molecules which produce detectable changes in a substrate.

In one embodiment the antibody specifically binds to all or a portion of a MTP, FABP2, ANXA5, PDHA2, CDS1, or GK2 protein. As used herein, the phrase “specifically binds” refers to binding of, for example, an antibody to an epitope or antigen or antigenic determinant in such a manner that binding can be displaced or competed with a second preparation of identical or similar epitope, antigen or antigenic determinant.

Other techniques may be used to detect MTP, FABP2, ANXA5, PDHA2, CDS1, or GK2 protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. MTP, FABP2, ANXA5, PDHA2, CDS1, or GK2 antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of MTP, FABP2, ANXA5, PDHA2, CDS1, or GK2 in a biological sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

In addition, the MTP, FABP2, ANXA5, PDHA2, CDS1, or GK2 protein may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. patent application Ser. Nos.: 20030199001, 20030134304, 20030077616, which are herein incorporated by reference.

Diagnostics and Prognostics

The present invention is directed to methods for diagnosis and prognosis of metabolic syndrome in a patient. The methods involve detecting differences in genes or gene products in a test sample obtained from a patient suspected of having metabolic syndrome and comparing the observed results (i.e. detection of the presence of a difference), to the result of at least one, preferably two, most preferably three of MTP, FABP2, ANXA5, PDHA2, CDS1, and GK2 found in a normal control sample. The difference in gene or gene product of MTP, FABP2, ANXA5, PDHA2, CDS1, and GK2 from that which is observed in a control sample is diagnostic and/or prognostic of metabolic syndrome. The levels of MTP, FABP2, ANXA5, PDHA2, CDS1, and GK2 can be represented by arbitrary units, for example as units obtained from a densitometer, luminometer, or an ELISA plate reader.

As used herein, the term “test sample” refers to a biological sample obtained from a patient to be tested for metabolic syndrome.

As used herein, a “biological sample” refers to a sample of biological material obtained from a patient, preferably a human patient, including a tissue, a tissue sample, a cell sample (e. g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), and a tumor sample. Biological samples can also be biological fluid samples e.g., blood, cerebral spinal fluid (CSF), or urine.

As used herein, the term “wild type or normal control sample” refers to a biological sample obtained from a “normal” or “healthy” individual that does not have metabolic syndrome.

For purposes of comparison, the test sample and normal control sample are of the same type, that is, obtained from the same biological source. The normal control sample can also be a standard sample that contains either the same concentration of MTP, FABP2, ANXA5, PDHA2, CDS1, and GK2 that is normally found in a biological sample of the same type and that is obtained from a healthy individual. Alternatively, for changes in nucleic acid determination, the normal control sample may be a nucleic acid obtained from a person who does not have metabolic syndrome.

The methods of the invention can also be practiced, for example, by selecting a combination of a MTP, FABP2, ANXA5, PDHA2, CDS1, or GK2 and one or more biomarkers for which changes correlate with metabolic syndrome. Example of metabolic syndrome biomarkers include, elevated triglyceride (TG) levels and levels of high density lipoprotein (HDL). Those skilled in the art will be able to select useful diagnostic and/or prognostic markers for detection in combination with the analysis of MTP, FABP2, ANXA5, PDHA2, CDS1, or GK2. Similarly, three or more, four or more or five or more or a multitude of biomarkers can be used together for determining a diagnosis or prognosis of a patient.

Treatment Methods

Also encompassed in the methods of the present application are methods to treat metabolic syndrome. Methods of treatment include, for example, regulators or modulators such as agonists and antagonists, partial agonists, inverse agonists, activators, co-activators and inhibitors of MTP, FABP2, ANXA5, PDHA2, CDS1, or GK2.

In one embodiment, metabolic syndrome is treated with antagonists of MTP, FABP2, ANXA5, PDHA2, CDS1, or GK2 by directly blocking the activity of the protein. This can be accomplished by a range of different approaches, including the use of antibodies, small molecules, and antagonists. MTP, FABP2, ANXA5, PDHA2, CDS1, or GK2 expression may also be inhibited in vivo by the use of antisense technology. Gene expression can be controlled through triple-helix formation or antisense DNA or RNA, both of which methods are based on binding of a polynucleotide to DNA or RNA. An antisense nucleic acid molecule which is complementary to a nucleic acid molecule encoding MTP, FABP2, ANXA5, PDHA2, CDS1, or GK2 can be designed based upon the isolated nucleic acid molecules encoding MTP, FABP2, ANXA5, PDHA2, CDS1, or GK2 by means known to those in the art. RNAi technology can also be used. RNA interference or “RNAi” is a term initially coined by Fire and co-workers to describe the observation that double-stranded RNA (dsRNA) can block gene expression when it is introduced into worms (Fire et al. (1998) Nature 391, 806-811). Isolated RNA molecules specific to MTP, FABP2, ANXA5, PDHA2, CDS1, or GK2 mRNA, which mediate RNAi, are antagonists useful in the method of the present invention. See for example U.S. patent application Ser. Nos.: 20030153519A1; 20030167490A1; and U.S. Pat. Nos.: 6,506,559; 6,573,099, which are herein incorporated by reference in their entirety.

In an alternative embodiment, MTP, FABP2, ANXA5, PDHA2, CDS1, or GK2 agonists, partial agonists, inverse agonists, activators, or co-activators may be used to treat metabolic syndrome.

The agonists, or antagonists of the invention are administered orally, topically, or by parenteral means, including subcutaneous and intramuscular injection, implantation of sustained release depots, intravenous injection, intranasal administration, and the like. Accordingly, agonists or antagonists of the invention may be administered as a pharmaceutical composition comprising the agonist or antagonist in combination with a pharmaceutically acceptable carrier. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol Registered TM, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, Silvadene Registered TM (Marion), Aquaphor Registered TM (Duke Laboratories), and the like. Other topical formulations include aerosols, bandages, and other wound dressings. Alternatively one may incorporate or encapsulate the compounds in a suitable polymer matrix or membrane, thus providing a sustained-release delivery device suitable for implantation near the site to be treated locally. Other devices include indwelling catheters and devices such as the Alzet Registered TM minipump. Ophthalmic preparations may be formulated using commercially available vehicles such as Sorbi-care Registered TM (Allergan), Neodecadron Registered TM (Merck, Sharp & Dohme), Lacrilube Registered TM, and the like, or may employ topical preparations such as that described in U.S. Pat. No. 5,124,155, incorporated herein by reference. Further, one may provide an antagonist in solid form, especially as a lyophilized powder. Lyophilized formulations typically contain stabilizing and bulking agents, for example human serum albumin, sucrose, mannitol, and the like. A thorough discussion of pharmaceutically acceptable excipients is available in Remington's Pharmaceutical Sciences (Mack Pub. Co.).

The amount of agonist or antagonist required to treat any metabolic syndrome will of course vary depending upon the nature and severity of the disorder, the age and condition of the subject, and other factors readily determined by one of ordinary skill in the art. Routes and frequency of administration, as well as dosage, will vary from individual to individual.

This invention also contemplates the use of competitive drug screening assays in which neutralizing antibodies capable of specifically binding polymorphic MTP, FABP2, ANXA5, PDHA2, CDS1, or GK2 polypeptide compete with a test compound for binding to the MTP, FABP2, ANXA5, PDHA2, CDS1, or GK2 polypeptide or fragments thereof. In this manner, the antibodies can be used to detect the presence of any peptide which shares one or more antigenic determinants of the MTP, FABP2, ANXA5, PDHA2, CDS1, or GK2 polypeptide.

A further technique for drug screening involves the use of host eukaryotic cell lines or cells which have a nonfunctional MTP, FABP2, ANXA5, PDHA2, CDS1, or GK2 genes. These host cell lines or cells are defective at the MTP, FABP2, ANXA5, PDHA2, CDS1, or GK2 polypeptide level. The host cell lines or cells are grown in the presence of drug compound. The rate of growth of the host cells is measured to determine if the compound is capable of regulating the growth of MTP, FABP2, ANXA5, PDHA2, CDS1, or GK2 defective cells.

KITS

The method lends itself readily to the formulation of kits which can be used in diagnosis and/or prognosis. Such a kit would comprise a carrier being compartmentalized to receive in close confinement one or more containers wherein a first container may contain a DNA fragment (either probe or primers) containing sequences for a given nucleic acid; i.e., an STS (short tandem repeat) or SNP (single nucleotide polymorphism) in MTP, FABP2, ANXA5, PDHA2, CDS1, or GK2, which are linked to metabolic syndrome. A second container may contain a different set of sequences for a second STS or SNP linked to metabolic syndrome. Other containers may contain reagents useful in the identification of nucleic acid changes, such as DNA polymerase, deoxynucleotide triphosphates, and enzyme substrates, reagents useful in PCR. Still other containers may contain restriction enzymes, buffers instructions, quality control materials, standards and the like. Instructions for using the method can also be part of the kit, whether in a container or as a package insert.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site is occupied by two different nucleotides.

### EXAMPLE 1

In order to establish the genetic linkage or connection between the desired polymorphism and the metabolic syndrome gene, it is preferable to analyze a set of familial relatives of the subject under investigation. The set is chosen so that it will allow determination of whether the metabolic syndrome phenotype is linked to the presence of the polymorphism. Thus, preferably, several individuals are examined. These may include an unaffected parent, an affected parent, an affected sibling, an unaffected sibling, as well as other, perhaps more distant, members. Ideally, an unaffected parent, an affected parent and an affected sibling should be utilized. If an affected parent is deceased, satisfactory results can still be obtained if unambiguous segregation of the polymorphism with the metabolic syndrome gene can be demonstrated in other members.

In one preferred embodiment one would look at multiple markers associated with susceptibility to metabolic syndrome. Thus, we recommend always looking at the polymorphic markers on 3q27, 17p12, glucocorticoid receptor gene located on 5q31-q33 and a microsomal triglyceride transfer protein (MTP) located on chromosome 4q22-q24, could be analyzed. The more polymorphisms seen at multiple locations, the greater the risk of susceptibility to metabolic syndrome.

In this example, a large Turkish family with 35 members suffering from metabolic syndrome and diagnosed with strict diagnostic criteria including triglyceride (TG) levels and levels of high density lipoprotein (HDL) were examined.

The individuals were diagnosed as affected with metabolic syndrome if their triglyceride (TG) levels were equal or greater than the 90^{th} percentile for age and sex, HDL equal or lower than 30 mg/dL for males and 34 mg/dL for females, and BMI≦30 kg/m²; unaffected if their TG levels equal or lower the 50^{th} percentile for their age and sex and HDL≧37 mg/dL for men and ≧42 mg/dL for women and BMI≦30 kg/m² and normoglycemic; and having an unknown affection status if no laboratory values were available or if their TG was between the 50^{th} and the 90^{th} percentile, and HDL levels were between the affected and unaffected status.

To identify the locus, the non-parametric logarithm of odds (lod) (NPL) scores analysis was performed. Because of theoretical difficulties concerning the application of the parametric lod score method to complex disease, and because there have been cases where the lod score method has appeared to produce erroneous results, a number of methods of linkage analysis have been developed which are broadly described as nonparametric.

The classical lod score method of linkage analysis has been very successful in mapping Mendelian disease genes and DNA markers. However in order to calculate a lod score it is necessary for the mode of transmission of all the loci involved to be fully specified, namely the disease allele frequencies and penetrance values of all the markers and phenotypes should be known or fairly accurately estimated.

It is possible to analyze diseases with complex (non-Mendelian) inheritance if the values for these parameters are known. Thus, one may specify particular risks for a genetically normal subject to be a phenocopy and for a genetically abnormal subject to be a non-penetrant carrier. However, when transmission is non-Mendelian it can be extremely difficult to estimate penetrance values, including phenocopy risks, and the allele frequencies of the disease mutation. Indeed, different mutations at different loci are likely to have different kinds of effects on susceptibility. For example, some mutations may cause major and some minor susceptibility and some may operate as dominant and some as recessive traits.

If different modes of transmission are operative in different families, or if different loci interact in the same family, then no one transmission model may be appropriate. It has therefore been argued that if the transmission model for a lod score analysis is specified incorrectly, the results produced assuming this model will not be valid and hence the lod score method should not be relied upon when analyzing a disease with unknown mode of inheritance. Therefore, a variety of methods have been developed to test for linkage without the need to specify values for the parameters defining the transmission model, and these methods are generally termed “nonparametric.” Such tests may also be termed “model-free”, implying that they may be applied without regard to the true transmission model. The NPL score can be analyzed using computer software programs such as GENEHUNTER (Kruglyak et al. Am J Hum Genet 1996 Jun;58(6):1347-1363).

We found the genetic markers useful according to the present invention to include polymorphic markers in the chromosomal region flanked by D4S2391 and D4S2394. The chromosomal region including these markers covers about 35 cM at a map position 93.4-129.9 cM.

Marker D4S2361 (also known as CHLC.ATA2AO3, ATA2A03, RH28026) is amplified with a forward primer: CCACGTGACTTTCATTAGGG (SEQ ID NO.: 1) and a reverse primer: ACACCATCATGGCGCATG (SEQ ID NO.: 2). The PCR product size varies between 152-153 (bp) (Homo sapiens GenBank Accession No.: G08322).

Marker D4S2394 (also known as RH28030, CHLC.ATA26BO8) is amplified with a forward primer: ACTGGTATGTCCTAACCCCC (SEQ ID NO.: 3) and a reverse primer: GATCTGCAGTTGGATTCTGG (SEQ ID NO.: 4). The PCR product size varies between 253-254 (bp) (Homo sapiens GenBank Accession: G08318).

This about 35 cM interval also includes about 83 genes that are listed on the Table 1. All of these genes can be used to analyze polymorphisms that may be associated with metabolic syndrome.

TABLE 1
Genes located in the map position 93.4-129.9M (LocusLink)

| | |
|---|---|
| DKFZp762K2 | hypothetical protein DKFZp762K2015 |
| PGDS | prostaglandin D2 synthase, hematopoietic |
| LIM | LIM protein (similar to rat protein kinase C-binding enigma) |
| BMPR1B | bone morphogenetic protein receptor, type IB |
| UNC5C | unc-5 homolog B (C. elegans) |
| PDHA2 | pyruvate dehydrogenase (lipoamide) alpha 2 |
| RAP1GDS1 | RAP1, GTP-GDP dissociation stimulator 1 |
| EIF4E | eukaryotic translation initiation factor 4E |
| METAP1 | methionyl aminopeptidase 1 |
| ADH5 | alcohol dehydrogenase 5 (class III), chi polypeptide |
| ADH4 | alcohol dehydrogenase 4 (class II), pi polypeptide |
| ADH1A | alcohol dehydrogenase 1A (class I), alpha polypeptide |
| ADH1B | alcohol dehydrogenase IB (class I), beta polypeptide |
| ADH1C | alcohol dehydrogenase 1C (class I), gamma polypeptide |
| ADH7 | alcohol dehydrogenase 7 (class IV), mu or sigma polypeptide |
| MTP | microsomal triglyceride transfer protein (large polypeptide, 88 kD) |
| DAPP1 | dual adaptor of phosphotyrosine and 3-phosphoinositides |
| H2AFZ | H2A histone family, member Z |
| LOC51705 | endomucin-2 |
| PPP3CA | protein phosphatase 3 (formerly 2B), catalytic subunit, alpha isoform |
| | (calcineurin A alpha) |
| BANK | hypothetical protein FLJ20706 |
| LOC64116 | up-regulated by BCG-CWS (bacillus Calmette-Guerin cell wall skeleton) |
| MANBA | mannosidase, beta A, lysosomal |
| NFkB1 | nuclear factor of kappa light polypeptide gene enhancer in B-cells 1 |
| | (p105) |
| UBE2D3 | ubiquitin-conjugating enzyme E2D 3 (UBC4/5 homolog, yeast) |
| LOC56898 | oxidoreductase UCPA |
| CENPE | centromere protein E (312 kD) |
| FLJ20032 | hypothetical protein FLJ20032 |
| KIAA1546 | KIAA1546 protein |
| LOC57117 | hypothetical nuclear factor SBBI22 |
| SID6-306 | inorganic pyrophosphatase |
| DKK2 | dickkopf homolog 2 (Xenopus laevis) |
| RAC1 | ras-related C3 botulinum toxin substrate 1 (rho family, small GTP binding |
| | protein Rac1 |
| SCYE1 | small inducible cytokine subfamily E, member 1 (endothelial monocyte- |
| | activating) |
| PAPSS1 | 3′-phosphoadenosine 5′-phosphosulfate synthase 1 |
| LEF1 | lymphoid enhancer-binding factor 1 |
| AGXT2L1 | alanine-glyoxylate aminotransferase 2-like 1 |
| LOC84570 | collagen-like Alzheimer amyloid plaque component precursor |
| FLJ20647 | hypothetical protein FLJ20647 |
| PLA2G12 | group XII secreted phospholipase A2 |
| SEC24B | SEC24 related gene family, member B (S. cerevisiae) |
| EGF | epidermal growth factor (beta-urogastrone) |
| ENPEP | glutamyl aminopeptidase (aminopeptidase A) |
| LCE | hypothetical protein MGC5487 |
| PITX2 | paired-like homeodomain transcription factor 2 |
| FABP2 | fatty acid binding protein 2, intestinal |
| LOC112881 | similar to hypothetical protein PRO0971 |
| T2BP | hypothetical protein MGC20791 |
| FLJ22670 | hypothetical protein FLJ22670 |
| LOC152625 | hypothetical gene supported by AL117508 |
| LOC152624 | similar to KIAA0737 gene product (H. sapiens) |
| LOC91431 | similar to prematurely terminated mRNA decay factor-like protein |
| ANK2 | ankyrin 2, neuronal |
| FLJ23548 | hypothetical protein FLJ23548 |
| NDST4 | N-deacetylase/N-sulfotransferase 4 |
| LOC152513 | similar to uridine 5′ monophosphate hydrolase 1 (H. sapiens |
| LOC152514 | similar to uridine 5′ monophosphate hydrolase 1 (H. sapiens) |
| NDST3 | N-deacetylase/N-sulfotransferase (heparan glucosaminyl) 3 |
| FABP2 | fatty acid binding protein 2, intestinal |
| KIAA1350 | KIAA1350 protein |
| LOC152980 | similar to KIAA0470 gene product (H. sapiens) |
| LOC152979 | similar to ribosomal protein L41 (H. sapiens) |
| LOC152977 | similar to N-deacetylase/N-sulfotransferase (heparan glucosaminyl) 3 (H. sapiens) |
| LOC152974 | similar to hypothetical protein |
| MAD2L1 | MAD2 mitotic arrest deficient-like 1 (yeast) |
| LOC116403 | similar to putative Listeria-induced protein LIND |
| LOC166906 | similar to KIAA1191 protein; hypothetical protein FLJ21022 |
| CCNA2 | cyclin A2 |
| LOC132332 | hypothetical gene LOC132332 |
| LOC152500 | similar to angiotensin receptor-like 2 (H. sapiens) |
| IL2 | interleukin 2 |
| LOC132612 | similar to unnamed protein product |
| KIAA1109 | KIAA1109 protein |
| LOC166378 | LOC166379 |
| FGF2 | fibroblast growth factor 2 (basic) |
| SPRY1 | sprouty homolog 1, antagonist of FGF signaling (Drosophila) |
| LOC166837 | LOC166837 |
| KIAA1223 | KIAA1223 protein |
| FLJ23056 | hypothetical protein FLJ23056 |
| LOC132362 | hypothetical protein LOC132815 |
| KIAA1284 | KIAA1284 protein |
| LOC152734 | similar to ribosomal protein L21 (H. sapiens) |
| LOC152736 | similar to 60S ribosomal protein L21 |
| STK18 | serine/threonine kinase 18 |

For example, the gene encoding the large subunit of the heterodimeric microsomal triglyceride transfer protein (MTP), is located in this chromosomal region. Protein disulfide isomerase (PDI) completes the heterodimeric microsomal triglyceride transfer protein, which has been shown to play a central role in lipoprotein assembly. Certain nonsense and frameshift mutations in the MTP have been shown to cause abetalipoproteinemia, a rare autosomal recessive disease characterized by a defect in assembly or secretion of plasma lipoproteins that contain apolipoprotein B (Wetterau et al., 1992). In general, MTP catalyzes the transport of triglyceride, cholesteryl ester, and phospholipid between phospholipid surfaces.

A gene encoding a component of the heterodimeric NFkB transcription factor also localizes in this chromosomal region. The NFkB complex has been shown to regulate the expression of inflammatory and immune genes. NFkB has been detected in numerous cell types that express cytokines, chemokines, growth factors, cell adhesion molecules, and some acute phase proteins in health and in various disease states. Inappropriate activation of NFkB has been linked to inflammatory events associated with for example atherosclerosis. Aljada et al. investigated whether insulin inhibits the proinflammatory chemokine monocyte chemoattractant protein-1 (MCP1), which attracts leukocytes to inflamed sites and is regulated by NFKB (Aljada et al., J Clin Endocrinol Metab. 2001 Jul;86(7):3250-6). The authors concluded that insulin at physiologically relevant concentrations exerts an inhibitory effect on the cardinal proinflammatory transcription factor NFkB and the proinflammatory chemokine MCP1. These effects were proposed to suggest an anti-inflammatory and potential anti-atherogenic effect of insulin-NFkB pathway.

A gene encoding PLA2G12, group XII secreted phospholipase A2 (PLA2G12) is also located at this chromosomal region. Secreted phospholipases A₂ (sPLA₂)¹ are Ca²⁺-dependent disulfide-rich 14-18-kDa enzymes that catalyze the hydrolysis of phospholipids at the sn 2-position to release fatty acids and lysophospholipids (Yuan et al., Biochim. Biophys. Acta 1441, 215-222, 1999; Gelb et al., Annu. Rev. Biochem. 64, 653-688, 1995; Balsinde et al., Annu Rev. Pharmacol. Toxicol. 39, 175-189). It is expressed as several transcripts including a major one of ˜1.4 kilobase, which is abundant in heart, skeletal muscle, and kidney. PLA2G12 transcripts are also present at lower levels in brain, liver, small intestine, lung, and placenta, and expressed poorly, if at all, in colon, thymus, spleen, and peripheral blood leukocytes. Transcripts can also be found in ovaries, testis, and prostate. (Gelb et al. J Biol Chem. 2000 Dec 22;275(51):39823-6.)

Another example of a candidate gene located in this chromosomal region is the gene encoding an intestinal fatty acid binding protein 2 (FABP2). The intracellular fatty acid-binding proteins (FABPs) belong to a multigene family with nearly twenty identified members. FABPs are divided into at least three distinct types, namely the hepatic-, intestinal- and cardiac-type. They form 14-15 kDa proteins and are thought to participate in the uptake, intracellular metabolism and/or transport of long-chain fatty acids. They may also be responsible in the modulation of cell growth and proliferation. Intestinal fatty acid-binding protein 2 gene contains four exons and is an abundant cytosolic protein in small intestine epithelial cells. This gene has a polymorphism at codon 54 that identified an alanine-encoding allele and a threonine-encoding allele. The Thr-54 protein is associated with increased fat oxidation and insulin resistance.

In addition to genes encoding a protein with known function, the genes encoding proteins of unknown function are candidates that are analyzed for their potential to harbor metabolic syndrome susceptibility mutations.

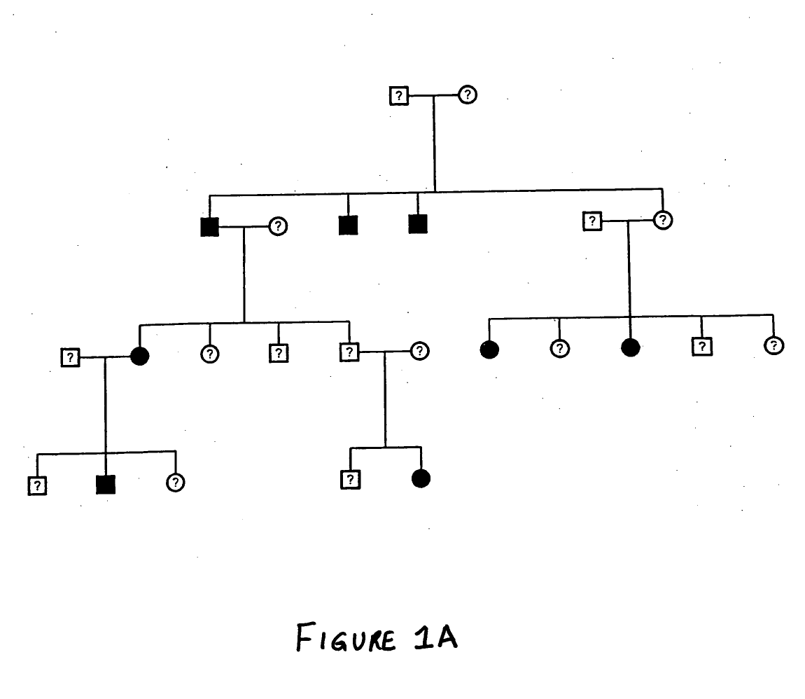

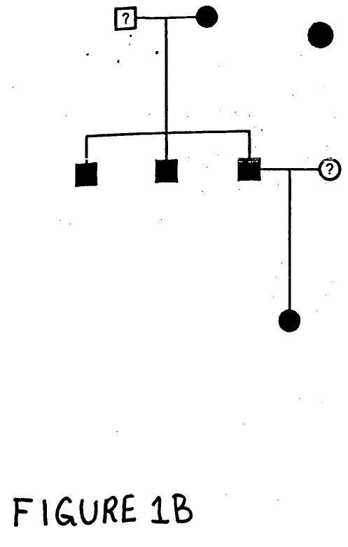

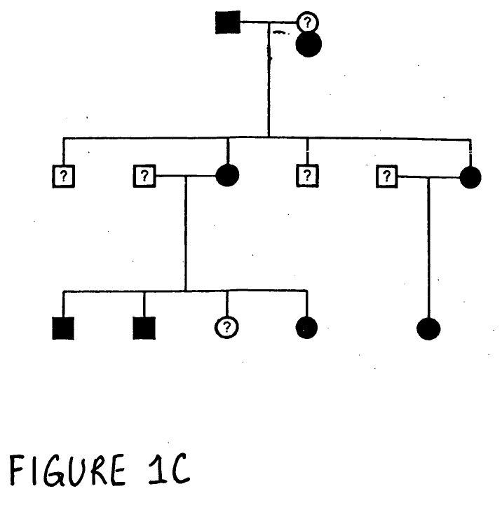

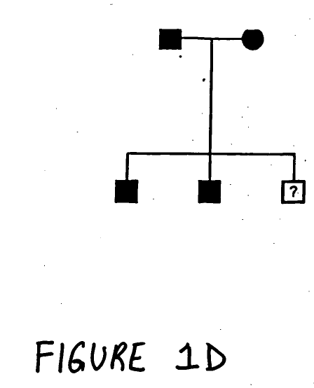

FIGS. 1A-1D show examples of the sub-pedigrees selected from analysis from the large Turkish pedigree with total of 170 members. 35 affected and 23 unaffected individuals were included into the genotype analysis using non-parametric lod score method to reveal the metabolic syndrome susceptibility locus between markers D4S2391 and D4S2394.

Blood samples were collected and DNA was isolated from the white blood cells of the living metabolic syndrome patients using standard methods known to one skilled in the art. Analysis of a limited number of closely related metabolic syndrome patients within our pedigree has now uncovered a new locus to look at in determining predisposition to metabolic syndrome susceptibility. This locus is on chromosome 4q. Furthermore, the metabolic syndrome susceptibility gene is located in a region covering about 35 cM between and including markers D4S2391 and D4S2394. This region contains at least about 83 identified genes.

Thus, one can look at polymorphisms withithihis region to determine susceptibility to metabolic syndrome.

Non-parametric linkage analysis was applied to 35 affected family members using GENEHUNTER and about 400 microsatellite markers covering the whole genome in about 10 cM intervals. The results from the multipoint analysis with markers covering the critical region in chromosome 4q are shown in Table 2.

TABLE 2
Multipoint Analysis of Markers on Chromosome 4q.
I. MULTIPOINT ANALYSIS

| Position | | | |
|---|---|---|---|
| (in cM | | | |
| from pter) | LOD Score | NPL Score | P-value |
| 95.46 | −0.39902 | 0.78971 | 0.157687 |
| 98.06 | 1.043668 | 1.12976 | 0.081292 |
| 100.66 | 1.519192 | 1.62074 | 0.04554 |
| 103.27 | 1.823667 | 2.28369 | 0.023506 |
| 105.87 | 2.052128 | 3.15116 | 0.004995 |
| 108.47 | 2.237737 | 4.26955 | 0.004112 |
| 110.48 | 2.299165 | 4.64523 | 0.003914 |
| 112.49 | 2.358298 | 5.06179 | 0.003906 |
| 114.5 | 2.416216 | 5.52826 | 0.003906 |
| 116.51 | 2.473674 | 6.05509 | 0.003906 |
| 118.52 | 2.531193 | 6.65462 | 0.003778 |
| 120.53 | 2.482607 | 6.57442 | 0.003855 |
| 122.53 | 2.428626 | 6.53787 | 0.003881 |
| 124.54 | 2.3663 | 6.54658 | 0.003855 |
| 126.55 | 2.290655 | 6.60321 | 0.003846 |
| 128.55 | 2.192495 | 6.71162 | 0.003743 |
| 129.61 | 2.01854 | 5.33285 | 0.003906 |
| 130.66 | 1.800166 | 3.9673 | 0.004245 |
| 131.71 | 1.504469 | 2.60736 | 0.009826 |
| 132.77 | 1.036306 | 1.24546 | 0.069719 |
| 133.82 | −1.515859 | −0.12601 | 0.46969 |
| 136.83 | −0.687998 | −0.04741 | 0.442482 |
| 139.84 | −0.545313 | −0.00856 | 0.430074 |
| 142.85 | −0.575065 | −0.00919 | 0.43029 |
| 145.86 | −0.773328 | −0.05086 | 0.44388 |

The p-values for the multipoint NPL analysis in this chromosomal area were 0.03 when the family was subdivided into 2 subfamilies and 0.004 when the family was subdivided in 4 subfamilies (see Table 2).

The single-point Maximum Lod Score using the software GAS (version 2.0) reported LOD scores ˜2.2 for the markers located in 4q25. No other chromosome reached this level of significance.

All the references described herein and throughout the specification are incorporated herein by reference in their entirety.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A method for screening an individual for susceptibility to and/or diagnosis of metabolic syndrome comprising:
obtaining a biological sample from an individual and analyzing a group of genes and/or gene products comprising microsomal triglyceride transfer protein (MTP), fatty acid binding protein 2 (FABP2), annexin A5 (ANXA5), pyruvate dehydrogenase (lipoamide) alpha 2 (PDHA2), CDP-diacylglycerol synthase (phosphatidate cytidylyltransferase) 1 (CDS1), and glycerol kinase 2 (GK2), wherein the analysis comprises comparing said individual's genes and/or gene products to the same group of genes and/or gene products from a control, wherein a difference in at least two genes or gene products between the individual and the control indicates that said individual is at risk for, or has, metabolic syndrome.

2. The method of claim 1, wherein at least three genes or gene products are analyzed.

3. The method of claim 1, wherein at least four genes or gene products are analyzed.

4. The method of claim 1, wherein at least five genes or gene products are analyzed.

5. The method of claim 1, wherein the analysis comprises analyzing nucleic acid.

6. The method of claim 5, wherein the nucleic acid is analyzed for one or more deletions or substitutions.

7. The method of claim 5, wherein the nucleic acid is analyzed for short tandem repeats (STRs) or single nucleotide polymorphisms (SNPs).

8. The method of claims 5, wherein the nucleic acid is analyzed for modifications.

9. The method of claim 8, wherein the modification is methylation.

10. The method of claim 5, wherein the nucleic acid is analyzed by a nucleic acid sequence determination assay or PCR.

11. The method of claim 1, wherein the gene product is analyzed.

12. The method of claim 1 1, wherein the gene product is mRNA or protein.

13. The method of claim 12, wherein the analysis is performed using antibodies directed against one or more of the gene products or antigenic fragments thereof.

14. The method of claim 12, wherein the gene product is analyzed for expression levels.

15. The method of claim 14, wherein an increased gene product expression in a biological sample obtained from said individual, as compared to the control, is indicative of an individual who is at risk for, or has, metabolic syndrome.

16. The method of claim 14, wherein decreased gene product expression in a biological sample obtained from said individual, as compared to the control, is indicative of an individual who is at risk for, or has, metabolic syndrome.

17. A method for treating metabolic syndrome comprising administering to a patient in need thereof an agent or compound that regulates the activity of MTP, FABP2, ANXA5, PDHA2, CDS1 or GK2.

18. The method of claim 17, wherein the agent or compound that regulates the activity of MTP, FABP2, ANXA5, PDHA2, CDS1, or GK2 is an inhibitor.

19. The method of claim 18, wherein the inhibitor is selected from the group consisting of an antibody or antibody fragment, small molecule, antisense nucleic acid, RNAi, siRNA, PNA, or aptamer.

20. The method of claim 17 wherein the agent or compound that regulates the activity of MTP, FABP2, ANXA5, PDHA2, CDS1, or GK2 is an activator.

21. The method of claim 20 wherein the activator is selected from the group consisting of a small molecule, partial agonists, inverse agonist, activator, or co-activator.


SEQUENCE LISTING (10 sequences)

SEQ ID NO: 1 - DNA, 20 bp, Artificial Sequence
  Description of Artificial Sequence Synthetic primer
ccacgtgactttcattaggg

SEQ ID NO: 2 - DNA, 18 bp, Artificial Sequence
  Description of Artificial Sequence Synthetic primer
acaccatcatggcgcatg

SEQ ID NO: 3 - DNA, 20 bp, Artificial Sequence
  Description of Artificial Sequence Synthetic primer
actggtatgtcctaaccccc

SEQ ID NO: 4 - DNA, 20 bp, Artificial Sequence
  Description of Artificial Sequence Synthetic primer
gatctgcagttggattctgg

SEQ ID NO: 5 - DNA, 2685 bp, Homo sapiens
atgattcttcttgctgtgctttttctctgcttcatttcctcatattcagcttctgttaaaggtcacacaactggtctctcattaaataatgaccggctgtacaagctcacgtactccactgaagttcttcttgatcggggcaaaggaaaactgcaagacagcgtgggctaccgcatttcctccaacgtggatgtggccttactatggaggaatcctgatggtgatgatgaccagttgatccaaataacgatgaaggatgtaaatgttgaaaatgtgaatcagcagagaggagagaagagcatcttcaaaggaaaaagcccatctaaaataatgggaaaggaaaacttggaagctctgcaaagacctacgctccttcatctaatccatggaaaggtcaaagagttctactcatatcaaaatgaggcagtggccatagaaaatatcaagagaggtctggctagcctatttcagacacagttaagctctggaaccaccaatgaggtagatatctctggaaattgtaaagtgacctaccaggctcatcaagacaaagtgatcaaaattaaggccttggattcatgcaaaatagcgaggtctggatttacgaccccaaatcaggtcttgggtgtcagttcaaaagctacatctgtcaccacctataagatagaagacagctttgttatagctgtgcttgctgaagaaacacacaattttggactgaatttcctacaaaccattaaggggaaaatagtatcgaagcagaaattagagctgaagacaaccgaagcaggcccaagattgatgtctggaaagcaggctgcagccataatcaaagcagttgattcaaagtacacggccattcccattgtggggcaggtcttccagagccactgtaaaggatgtccttctctctcggagctctggcggtccaccaggaaatacctgcagcctgacaacctttccaaggctgaggctgtcagaaacttcctggccttcattcagcacctcaggactgcgaagaaagaagagatccttcaaatactaaagatggaaaataaggaagtattacctcagctggtggatgctgtcacctctgctcagacctcagactcattagaagccattttggactttttggatttcaaaagtgacagcagcattatcctccaggagaggtttctctatgcctgtggatttgcttctcatcccaatgaagaactcctgagagccctcattagtaagttcaaaggttctattggtagcagtgacatcagagaaactgttatgatcatcactgggacacttgtcagaaagttgtgtcagaatgaaggctgcaaactcaaagcagtagtggaagctaagaagttaatcctgggaggacttgaaaaagcagagaaaaaagaggacaccaggatgtatctgctggctttgaagaatgccctgcttccagaaggcatcccaagtcttctgaagtatgcagaagcaggagaagggcccatcagccacctggctaccactgctctccagagatatgatctccctttcataactgatgaggtgaagaagaccttaaacagaatataccaccaaaaccgtaaagttcatgaaaagactgtgcgcactgctgcagctgctatcattttaaataacaatccatcctacatggacgtcaagaacatcctgctgtctattggggagcttccccaagaaatgaataaatacatgctcgccattgttcaagacatcctacgtttggaaatgcctgcaagcaaaattgtccgtcgagttctgaaggaaatggtcgctcacaattatgaccgtttctccaggagtggatcttcttctgcctacactggctacatagaacgtagtccccgttcggcatctacttacagcctagacattctctactcgggttctggcattctaaggagaagtaacctgaacatctttcagtacattgggaaggctggtcttcacggtagccaggtggttattgaagcccaaggactggaagccttaatcgcagccacccctgacgagggggaggagaaccttgactcctatgctggtatgtcagccatcctctttgatgttcagctcagacctgtcacctttttcaacggatacagtgatttgatgtccaaaatgctgtcagcatctggcgaccctatcagtgtggtgaaaggacttattctgctaatagatcattctcaggaacttcagttacaatctggactaaaagccaatatagaggtccagggtggtctagctattgatatttcaggtgcaatggagtttagcttgtggtatcgtgagtctaaaacccgagtgaaaaatagggtgactgtggtaataaccactgacatcacagtggactcctcttttgtgaaagctggcctggaaaccagtacagaaacagaagcaggcttggagtttatctccacagtgcagttttctcagtacccattcttagtttgcatgcagatggacaaggatgaagctccattcaggcaatttgagaaaaagtacgaaaggctgtccacaggcagaggttatgtctctcagaaaagaaaagaaagcgtattagcaggatgtgaattcccgctccatcaagagaactcagagatgtgcaaagtggtgtttgcccctcagccggatagtacttccagcggatggttttga

SEQ ID NO: 6 - DNA, 399 bp, Homo sapiens
atggcgtttgacagcacttggaaggtagaccggagtgaaaactatgacaagttcatggaaaaaatgggtgttaatatagtgaaaaggaagcttgcagctcatgacaatttgaagctgacaattacacaagaaggaaataaattcacagtcaaagaatcaagcgcttttcgaaacattgaagttgtttttgaacttggtgtcacctttaattacaacctagcagacggaactgaactcagggggacctggagccttgagggaaataaacttattggaaaattcaaacggacagacaatggaaacgaactgaatactgtccgagaaattataggtgatgaactagtccagacttatgtgtatgaaggagtagaagccaaaaggatctttaaaaaggattga

SEQ ID NO: 7 - DNA, 963 bp, Homo sapiens
atggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaa

SEQ ID NO: 8 - DNA, 1167 bp, Homo sapiens
atgctggccgccttcatctcccgcgtgttgaggcgagttgcccagaaatcagctcgcagagtgctggtggcatcccgtaactcctcaaatgacgctacatttgaaattaagaaatgtgatctttatctgttggaagagggtccccctgtcactacagtgctcactagggcggaggggcttaaatactacaggatgatgctgactgttcgccgcatggaattgaaggcagatcagctgtacaaacagaaattcattcgcggtttctgtcacctgtgcgatggtcaggaagcttgttgcgtgggccttgaggccggcataaacccctcggatcacgtcattacatcctatagggctcatggtgtgtgctatactcggggactttctgtccgatccattctcgcagagctgacgggaagaagaggaggttgtgctaaaggaaaaggaggatcgatgcatatgtataccaagaacttctatgggggcaatggcatcgtcggtgcacagggccccctgggcgctggcattgctctggcctgtaaatataaaggaaacgatgagatctgtttgactttatatggggatggcgctgcgaatcaggggcagatagccgaagctttcaatatggcagctttatggaaattaccttgtgttttcatctgtgagaataacctatatggaatgggaacatctactgagagagcagcagccagccctgattactacaagaggggcaattttatccctgggctaaaggtcgatggaatggatgttctgtgtgttcgtgaggcaacaaaatttgcagctaactactgtagatctggaaaggggcccatactgatggagctgcaaacctaccgttatcatggacacagtatgagtgatcctggagtcagttatcgtacacgagaagaaattcaggaagtaagaagtaagagggatcctataataattctccaagatagaatggtaaacagcaagctcgccactgtggaagaattaaaggaaattggggctgaggtgaggaaagaaattgatgatgctgcccagtttgctaccactgatcctgagccacatttggaagaattaggccatcacatctacagcagtgattcatcttttgaagttcgtggtgcaaatccatggatcaagtttaagtccgtcagttaa

SEQ ID NO: 9 - DNA, 1386 bp, Homo sapiens
atgttggagctgaggcaccggggaagctgccccggccccagggaagcggtgtcgccgccacaccgcgagggagaggcggccggcggcgaccacgaaaccgagagcaccagcgacaaagaaacagatattgatgacagatatggagatttggattccagaacagattctgatattccggaaattccaccatcctcagatagaacccctgagattctcaaaaaagctctatctggtttatcttcaaggtggaaaaactggtggatacgtggaattctcactctaactatgatctcgttgtttttcctgatcatctatatgggatccttcatgctgatgcttcttgttctgggcatccaagtgaaatgcttccatgaaattatcactataggttatagagtctatcattcttatgatctaccatggtttagaacactaagttggtactttctattgtgtgtaaactactttttctatggagagactgtagctgattattttgctacatttgttcaaagagaagaacaacttcagttcctcattcgctaccatagatttatatcatttgccctctatctggcaggtttctgcatgtttgtactgagtttggtgaagaaacattatcgtctgcagttttatatgttcgcatggactcatgtcactttactgataactgtcactcagtcacaccttgtcatccaaaatctgtttgaaggcatgatatggttccttgttccaatatcaagtgttatctgcaatgacataactgcttacctttttggatttttttttgggagaactccattaattaagttgtctcctaaaaagacttgggaaggattcattggtggtttcttttccacagttgtgtttggattcattgctgcctatgtgttatccaaataccagtactttgtctgcccagtggaataccgaagtgatgtaaactccttcgtgacagaatgtgagccctcagaacttttccagcttcagacttactcacttccaccctttctaaaggcagtcttgagacaggaaagagtgagcttgtaccctttccagatccacagcattgcactgtcaacctttgcatctttaattggcccatttggaggcttctttgctagtggattcaaaagagccttcaaaatcaaggattttgcaaataccattcctggacatggtgggataatggacagatttgattgtcagtatttgatggcaacttttgtacatgtgtacatcacaagttttatccggggcccaaatcccagcaaagtgctacagcagttgttggtgcttcaacctgaacagcagttaaatatatataaaaccctgaagactcatctcattgagaaaggaatcctacaacccaccttgaaggtataa

SEQ ID NO: 10 - DNA, 1662 bp, Homo sapiens
atggcagccccaaagacagcagctgtggggccgttggtgggagcggtggtccagggcaccaactccactcgctttctggttttcaattcaaaaacagcggaactacttagtcatcacaaagtggaattaacacaagagttcccaaaagaaggatgggtggaacaagaccctaaagaaattcttcagtctgtctacgagtgtatagcgagaacgtgtgagaaacttgacgaactgaatattgatatatccaacataaaagctgttggtgtcagcaatcagagggaaaccactgtaatctgggacaagttaacaggagagcctctctacaatgctgtggtgtggcttgatctaagaacccagactactgttgaggatcttagtaaaaaaattccaggaaatagtaacttcgtcaagtctaagacaggccttccactcagcacttacttcagtgcagtaaaacttcgttggatgcttgacaatgtgagaaacgtccaaaaggctgttgaagaaggtagagctctttttggtaccattgattcatggcttatctggagtttgacaggaggagttaatggaggcgtgcattgtacagatgtaacaaatgcaagtaggacaatgctttttaatatccattctttggaatgggataaagagctctgtgacttttttgaaattccaatggaccttcttccaaatgtcttcagttcttctgagatctatggcctaattaaaactggagccctggaaggtgtgccaatatctgggtgtttgggggaccaatgtgctgcattagtaggacaaatgtgcttccaggagggacaagccaaaaacacctatggaacaggttgcttcttactgtgtaatacgggtcgtaaatgtgtgttttctgaacatggccttttgaccacagtagcttacaaactaggcagagagaagccagcatattatgcactggaaggttctgttgctatagcaggtgctgttattcgttggctaagagacaatcttggaattatagagacctcaggagacattgaaagacttgctaaagaagtaggaacttcttatggctgttactttgtcccagccttttcagggttatatgcaccttattgggagcccagtgcaagagggatactctgtggcctcactcagtttaccaataaatgtcatattgcttttgctgcattagaagctgtttgtttccaaacccgagagattttggaagccatgaaccgtgactgtggaattccacttcgtcatttgcaggtagatggaggaatgaccaacaacaaagttcttatgcagctacaagcagatattcttcatattccagtaataaaaccctttatgcctgaaacaactgcactaggagctgccatggcagcaggggctgcagagggagtaagcgtttggagccttgaaccccaggctttgtcagttctcaggatggaacgatttgaaccacagatccaggccacagaaagtgaaattcgttatgccacatggaagaaagccgtaatgaagtcaatgggttgggttaccagtcagtctcctgaaggtggtgatccttctatcttctctagtctgcctttgggattttttatagtgagtagcatggtaatgctaattggagcaagatatatctcgggtgtgccataa